(19) United States
(12) Patent Application Publication
(10) Pub. No.: US 2007/0231280 A1
(43) Pub. Date: Oct. 4, 2007

(54) Ternary Metal Mixed Oxide Powder

(75) Inventors: Kai Schumacher, Hofheim (DE); Rainer Golchert, Dieburg (DE); Oswin Klotz, Westerngrund (DE); Uwe Diener, Grosskrotzenburg (DE); Steffen Hasenzahl, Hanau (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: Degussa AG, Duesseldorf (DE)

(21) Appl. No.: 11/569,415

(22) PCT Filed: Apr. 19, 2005
(86) PCT No.: PCT/EP05/04126

§ 371 Date: Nov. 20, 2006

(30) Foreign Application Priority Data
May 21, 2004 (DE) 10 2004 025 143.6

(51) Int. Cl. A61Q 17/04; A61K 8/25; C01B 33/26; C01B 35/00

(52) U.S. Cl. 424/59; 423/279; 423/327.1

(57) ABSTRACT

Ternary, pyrogenically prepared, crystalline metal mixed oxide powder having the components titanium and silicon and a third component chosen from the group comprising aluminium, zirconium, zinc, tin, magnesium, yttrium, vanadium, tungsten, tantalum, cerium or boron. It is prepared by a procedure in which a vaporizable or atomizable silicon and titanium compound and a vaporizable or atomizable compound of a third mixed oxide component are mixed with hydrogen and primary air and the gas mixture is burned into a reaction chamber, and the powder formed is separated off from the gaseous reaction products. It can be employed in sunscreen formulations.

The invention relates to a ternary metal mixed oxide powder having the components silicon and titanium, the preparation thereof and to a cosmetic formulation which comprises the ternary metal mixed oxide powder.

Titanium dioxide is used diversely in cosmetic formulations since it is chemically inert and toxicologically acceptable, and leads neither to skin irritations nor to sensitization.

It is furthermore known to employ mixed oxide powders containing titanium dioxide as a constituent of sunscreen formulations. DE-A-4235996 describes the preparation of silicon/titanium mixed oxide powders and aluminium/titanium mixed oxide powders by means of a flame hydrolysis process. The content of silicon dioxide or aluminium oxide in these is 1 to 20 wt. %.

EP-A-1197472 furthermore discloses an iron/silicon/titanium mixed oxide powder which is prepared by flame hydrolysis and can comprise in each case 0.5 to 50 wt. % iron oxide or silicon dioxide. This ternary mixed oxide powder indeed has a higher UV absorption than pure titanium dioxide, but is not an ideal constituent of a sunscreen formulation because of its intensive coloration. Moreover, experiments have shown that often it can be incorporated into sunscreen formulations only with difficulty.

Further ternary metal mixed oxide powders are described in DE-A-10233193. These are likewise obtained from a flame hydrolysis process, but are amorphous to x-rays and therefore less suitable as a constituent of sunscreen formulations.

The object of the invention is to provide a powder which shows a high UV absorption in sunscreen formulations in particular, is not or is only slightly coloured and can readily be incorporated into sunscreen formulations.

The invention provides a ternary, pyrogenically prepared, crystalline metal mixed oxide powder having the components titanium and silicon and a third component chosen from the group comprising aluminium, zirconium, zinc, tin, magnesium, yttrium, vanadium, tungsten, tantalum, cerium or boron.

In this context, pyrogenic is to be understood as meaning the processes of a flame oxidation, in which the starting substances of the metal mixed oxide powder are oxidized by means of an oxygen-containing gas, and a flame hydrolysis, in which the starting substances of the metal mixed oxide powder are hydrolysed. In between, there are mixed forms in which flame oxidation and flame hydrolysis exist simultaneously. The starting substances can be introduced into the flame in the form of gases or atomized solutions.

A metal mixed oxide powder produced by the flame hydrolysis route is preferred here, since a powder prepared by this route as a rule has a lower content of impurities.

The metal mixed oxide powder according to the invention comprises primary particles which have predominantly grown together to form aggregates. It can be seen in TEM photographs that some of the primary particles of a metal mixed oxide powder according to the invention comprise a core of titanium dioxide and a shell of a) silicon dioxide, b) the oxide of the third mixed oxide component or c) a mixed oxide of silicon and the third mixed oxide component. Another portion of the same powder comprises titanium dioxide, silicon dioxide and the third mixed oxide component as a constituent of a primary particle. The form in which the primary particles are present depends on the composition and the process parameters during the preparation.

In the context of the invention, crystalline is to be understood as meaning that the ternary metal mixed oxide powder according to the invention shows reflexes in an x-ray diffraction analysis. All the ternary metal mixed oxide powders according to the invention show the reflexes of rutile and anatase as titanium dioxide modifications. The x-ray diffraction diagrams can moreover show further reflexes which can be assigned, inter alia, to the third mixed oxide component. Reflexes of silicon dioxide cannot be detected in the ternary metal mixed oxide powders according to the invention. The silicon dioxide content accordingly is present in a form which is amorphous to x-rays.

Aluminium can be a preferred third mixed oxide component. Silicon/titanium/aluminium mixed oxide powders according to the invention are particularly suitable for the preparation of sunscreen formulations.

In addition to SiO₂, TiO₂ and the third mixed oxide component, the metal mixed oxide powder according to the invention can also comprise small amount of impurities from the starting substances or process-related impurities. In total, these impurities are less than 1 wt. %, as a rule less than 0.1 wt. %. In particular, the metal mixed oxide powder according to the invention can also contain chloride.

The degree of intergrowth of the primary particles of the metal mixed oxide powder according to the invention can be determined by means of DBP absorption (dibutyl phthalate absorption). In the DBP absorption, the power uptake, or the torque (in Nm), of the rotating paddles of the DBP measuring apparatus on addition of defined amounts of DBP is measured, in a manner comparable to a titration. For the ternary metal mixed oxide powder according to the invention, a sharply pronounced maximum with a subsequent drop at a particular addition of DBP can result here, or the DBP measuring instrument detects no maximum. In the latter case, the primary particles have a low degree of intergrowth.

Metal mixed oxide powders according to the invention which have no maximum in the DBP absorption may be advantageous, since they can readily be incorporated into formulations.

The metal mixed oxide powder according to the invention can preferably have a BET surface area of 10 to 200 m²/g, and particularly preferably one of 40 to 120 m²/g.

It may furthermore be preferable to have a content of titanium dioxide of 40 to 99, of silicon dioxide of 0.5 to 30 and of the third mixed oxide component of 0.5 to 30 wt. %. Particularly preferably, the content of titanium dioxide can be 60 to 95, of silicon dioxide can be 1 to 20 and of the third mixed oxide component can be 1 to 10 wt. %.

The rutile/anatase ratio of the metal mixed oxide powder according to the invention can be varied over wide limits, as a rule from 90:10 to 10:90. In this context, a particular ratio can be obtained independently of the BET surface area by modifying process parameters during the preparation.

It may be advantageous if the surface of the metal mixed oxide powder according to the invention is hydrophobized. The hydrophobization can be carried out by spraying the metal mixed oxide powder according to the invention with a hydrophobizing agent or a mixture of hydrophobizing agents, optionally in the presence of water, subsequently mixing the components for 15 to 30 minutes and then conditioning the mixture at a temperature of 100 to 500° C. over a period of 1 to 6 hours. All the hydrophobizing agents mentioned in EP-A-722992 can be employed for this, hexamethyldisilazane, trimethoxyoctylsilane, dimethylpolysiloxane and trimethoxypropylsilane being particularly preferred.

The invention also provides a process for the preparation of the metal mixed oxide powder according to the invention, which is characterized in that
vaporizable or atomizable starting substances of silicon and titanium and a vaporizable or atomizable starting substance of aluminium, zirconium, zinc, tin, magnesium, yttrium, vanadium, tungsten, tantalum, cerium or boron are vaporized and transferred by means of a carrier gas into the mixing chamber of a known burner, the vaporized or atomized, gaseous compounds being metered in a ratio which corresponds to the composition of the metal mixed oxide powder,
the gaseous compounds are mixed with hydrogen and primary air in the mixing chamber of the known burner and the gas mixture is burned into a reaction chamber, and
the powder formed is separated off from the gaseous reaction products and optionally freed from adhering reaction products by means of water vapour.

The starting substances can be vaporized together or separately. It is also possible for two components to be premixed.

The ratio of the hydrogen fed in to that required stoichiometrically is called gamma. Analogously, the ratio of the oxygen fed in to the oxygen required stoichiometrically is called lambda. Required stoichiometrically in this context means in each case the amount of hydrogen and oxygen exactly necessary for hydrolysis of the titanium/silicon halide. Accordingly:

gamma=H₂ fed in (mol)/stoichiometric H₂ (mol)

lambda=O₂ fed in (mol)/stoichiometric O₂ (mol)

In the preparation of the metal mixed oxide powder according to the invention, it has been found to be advantageous if gamma and lambda assume values between 1 and 10, values between 1 and 3 being particularly preferred for gamma and between 1 and 5 for lambda.

By varying the flame parameters lambda and gamma, it is possible to prepare metal mixed oxide powders having a variable rutile/anatase ratio with (approximately) the same BET surface area (see Examples 2 and 3 or 7 and 8).

The nature of the starting substances is not limited, as long as they can be vaporized, and oxidized or hydrolysed under the reaction conditions. Halides, nitrates or organometallic starting substances can preferably be employed. Chlorides are particularly preferred because of their availability and economic characteristics.

It may furthermore be advantageous, in addition to the primary air in the mixing chamber, to introduce air (secondary air) directly into the reaction chamber. The ratio of primary air/secondary air in this case is between 10 and 0.5.

It may also be advantageous if the flame burns into a reaction chamber which is closed off from the ambient air. By this means it is possible to meter the amount of secondary air precisely and thereby to optimize the process procedure. The vacuum which prevails in the reaction chamber is preferably between 5 and 80 mbar.

The invention also provides the use of the metal mixed oxide powder according to the invention for the preparation of cosmetics articles, in lacquers, as a catalyst, as a catalyst support, as a photocatalyst and as a UV absorber.

The invention also provides sunscreen compositions which comprise the metal mixed oxide particles according to the invention in an amount of 0.01 to 25 wt. %. In addition, the sunscreen composition according to the invention can be employed in mixtures with known inorganic UV-absorbing pigments and/or organic UV filters.

Possible known UV-absorbing pigments are titanium dioxides, zinc oxides, aluminium oxides, iron oxides, silicon dioxide, silicates, cerium oxides, zirconium oxides, barium sulfate or mixtures thereof.

Possible chemical UV filters are all the water- or oil-soluble UVA and also UV-B filters known to the expert, of which sulfonic acid derivatives of benzophenones and benzimidazoles, derivatives of dibenzoylmethane, benzylidenecamphor and derivatives thereof, derivatives of cinnamic acid and esters thereof, or esters of salicylic acid may be mentioned by way of example but non-limitatively.

The sunscreen compositions according to the invention can furthermore comprise the solvents known to the expert, such as water, mono- or polyhydric alcohols, cosmetics oils, emulsifiers, stabilizers, consistency regulators, such as carbomers, cellulose derivatives, xanthan gum, waxes, bentones, pyrogenic silicas and further substances which are conventional in cosmetics, such as vitamins, antioxidants, preservatives, dyestuffs and perfumes.

The sunscreen composition according to the invention can typically be in the form of an emulsion (O/W, W/O or multiple), aqueous or aqueous-alcoholic gel or oily gel, and be made available in the form of lotions, creams, milk sprays, mousses, as a stick or in other usual forms.

### EXAMPLES

Analysis:

The BET surface area is determined in accordance with DIN 66131. The content of SiO₂ and TiO₂ is determined by x-ray fluorescence analysis and/or chemical analysis.

The dibutyl phthalate absorption is measured with a RHEOCORD 90 apparatus from Haake, Karlsruhe. For this, 16 g of the metal oxides described, accurately to 0.001 g, are filled into a kneading chamber, this is closed with a lid and dibutyl phthalate is metered in via a hole in the lid at a predetermined metering rate of 0.0667 ml/s. The kneader is operated with a motor speed of 125 revolutions per minute. When the maximum torque is reached, the kneader and the metering of DBP are switched off automatically. The DBP absorption is calculated from the amount of DBP consumed and the amount of particles weighed out according to the formula: DBP number (ml/100 g)=(consumption of DBP in ml/particles weighed out in g)×100.

### Embodiment Examples

AlCl₃ (Example 11: ZrCl₄), SiCl₄ and TiCl₄ are vaporized separately according to the amounts stated in Table 1. The vapours are transferred by means of an inert gas into a mixing chamber. In this, they are mixed with hydrogen and dried air (primary air) (amounts according to Table 1) and the mixture is burned into a reaction chamber. In addition, secondary air can be fed into the reaction chamber (Examples 2, 3 and 10). In the coagulation zone, the powder is cooled to approx. 100° C. and then separated off with a filter. Adhering chloride is removed by treating the powder with damp air at temperatures of between 400° C. and 700° C.

The starting substances and amounts of the examples are reproduced in Table 1. The flame parameters and physico-chemical data of the metal mixed oxide powders are reproduced in Table 2.

Sunscreen Formulation

Phase A: Isolan GI 34 (3.0), castor oil (1.2), Tegesoft OP (10.0), Tegesoft Liquid (5.0), glycerol 86% (3.0), phase B: Paracera W80 (1.8), isohexadecane (5.0), phase C: metal mixed oxide powder according to the invention according to Example 2 (4.0), phase D: magnesium sulfate (0.5), completely demineralized water (66.5). (Values in parentheses in wt. %).

Phase A is heated to 70° C. in a mixer. After melting on a magnetic hot-plate at 80° C., phase B is added to phase A. Phase C is stirred into the oily phase at approx. 300 rpm and in vacuo. Phase D is also heated to 70° C., and added to the mixture of A-C in vacuo.

TABLE 1
Starting substances

| | | | | | Primary | Secondary | Carrier |
|---|---|---|---|---|---|---|---|
| | AlCl₃ | SiCl₄ | TiCl₄ | H₂ | air | air | gas |
| Example | kg/h | kg/h | kg/h | Nm³/h | Nm³/h | Nm³/h | Nm³/h |
| 1 | 0.1467 | 0.1527 | 1.3527 | 0.66 | 3.00 | — | 0.265 |
| 2 | 0.1401 | 0.1440 | 1.2640 | 0.66 | 3.30 | 1 | 0.265 |
| 3 | 0.1380 | 0.1466 | 1.3730 | 0.50 | 4.40 | 1 | 0.265 |
| 4 | 0.1465 | 0.1466 | 1.2867 | 0.66 | 2.00 | — | 0.265 |
| 5 | 0.1456 | 0.1466 | 1.3130 | 0.66 | 2.30 | — | 0.265 |
| 6 | 0.1496 | 0.1480 | 1.2940 | 0.66 | 2.20 | — | 0.265 |
| 7 | 0.0161 | 0.0880 | 1.3920 | 0.66 | 2.30 | — | 0.265 |
| 9 | 0.0966 | 0.0867 | 1.3800 | 0.66 | 2.80 | — | 0.265 |
| 8 | 0.0620 | 0.0800 | 1.3650 | 0.66 | 2.45 | — | 0.265 |
| 10 | 0.1798 | 0.4410 | 1.3600 | 0.66 | 2.90 | 1 | 0.265 |
| 11⁽*⁾ | 0.110 | 0.1251 | 1.3500 | 0.50 | 2.30 | — | 0.265 |

⁽*⁾ZrCl₄ instead of AlCl₃

TABLE 2
Flame parameters and analysis of powders

| | | | | | | | | DBF |
|---|---|---|---|---|---|---|---|---|
| | gamma | lambda | BET | Al₂O₃ | SiO₂ | TiO₂ | | number |
| Example | core | core | m²/g | wt. % | wt. % | wt. % | R/A | g/100 |
| 1 | 1.66 | 1.90 | 80 | 6.5 | 10.5 | 83.0 | — | — |
| 2 | 1.77 | 2.09 | 85 | 6.5 | 10.1 | 83.4 | 60/40 | n.d.⁽*⁾ |
| 3 | 1.26 | 3.68 | 91 | 6.4 | 10.0 | 83.6 | 35/65 | n.d. |
| 4 | 1.74 | 1.27 | 44 | 6.7 | 10.5 | 82.8 | 81/19 | — |
| 5 | 1.71 | 1.46 | 54 | 7.3 | 9.9 | 82.8 | — | — |
| 6 | 1.73 | 1.40 | 48 | 7.2 | 9.8 | 83.0 | — | n.d. |
| 7 | 1.85 | 1.46 | 45 | 3.4 | 6.5 | 91.1 | 74/26 | n.d. |
| 8 | 1.77 | 1.78 | 49 | 4.5 | 6.0 | 89.5 | 65/35 | n.d. |
| 9 | 1.84 | 1.55 | 45 | 3.1 | 5.6 | 91.4 | — | — |
| 10 | 1.37 | 1.84 | 49 | 8.4 | 19.5 | 71.9 | — | n.d. |
| 11 | 1.34 | 1.93 | 51 | 8.5 | 6.4 | 85.0 | — | n.d. |

⁽*⁾n.d. = cannot be determined

1. Ternary, pyrogenically prepared, crystalline metal mixed oxide powder having the components titanium and silicon and a third component selected from the group consisting of aluminium, zirconium, zinc, tin, magnesium, yttrium, vanadium, tungsten, tantalum cerium, boron and mixtures thereof.

2. Ternary, pyrogenically prepared, crystalline metal mixed oxide powder according to claim 1, wherein the metal mixed oxide powder is prepared by the flame hydrolysis route.

3. Ternary, pyrogenically prepared, crystalline metal mixed oxide powder according to claim 1 wherein the third component is aluminium.

4. Ternary, pyrogenically prepared, crystalline metal mixed oxide powder according to claim 1 wherein no maximum can be determined in the dibutyl phthalate absorption.

5. Ternary, pyrogenically prepared, crystalline metal mixed oxide powder according to claim 1 wherein the BET surface area is 10 to 200 m² g.

6. Ternary, pyrogenically prepared, crystalline metal mixed oxide powder according to claim 1 wherein the content of titanium dioxide is 40 to 99, of silicon dioxide is 0.5 to 30 and the content of the third component is 0.5 to 30 wt. %.

7. Ternary, pyrogenically prepared, crystalline metal mixed oxide powder according to claim 1 wherein the rutile/anatase ratio is from 90:10 to 10:90.

8. Ternary, pyrogenically prepared, crystalline metal mixed oxide powder according to claim 1 wherein the surface is hydrophobized.

9. Process for the preparation of the ternary, pyrogenically prepared, crystalline metal mixed oxide powder according to claim 1 wherein
vaporizable or atomizable compounds of silicon and titanium and a vaporizable or atomizable compound of aluminium, zirconium zinc, tin, magnesium, yttrium, vanadium, tungsten, tantalum, cerium or boron are vaporized and transferred by means of a carrier gas into the mixing chamber of a known burner, the vaporized or atomized, gaseous compounds being metered in a ratio which corresponds to the composition of the ternary metal mixed oxide powder,
the gaseous compounds are mixed with hydrogen and primary air in the mixing chamber of the known burner and the gas mixture is burned into a reaction chamber, and
the ternary metal mixed oxide powder formed is separated off from the gaseous reaction products and optionally freed from adhering reaction products by means of water vapour.

10. Process according to claim 9, wherein the vaporizable compounds are chlorides.

11. Process according to claim 9 wherein secondary air is additionally introduced into the reaction chamber.

12. Use of the ternary, pyrogenically prepared, crystalline metal mixed oxide powder according to claim 1 for the preparation of cosmetics articles, in lacquers, as a catalyst, as a catalyst support, as a photocatalyst and as a UV absorber.

13. Sunscreen formulation comprising the ternary, pyrogenically prepared, crystalline metal mixed oxide powder according to claim 1 wherein the content of the crystalline metal mixed oxide powder is between 0.01 and 25 wt. %, based on the amount of the sunscreen composition.
